(19) United States
(12) Patent Application Publication
(10) Pub. No.: US 2024/0277346 A1
(43) Pub. Date: Aug. 22, 2024

(54) BATTERY POWERED CORDLESS PNEUMATIC TOURNIQUET

(71) Applicant: Pieter Lagaay, Palo Cedro, CA (US)

(72) Inventor: Pieter Lagaay, Palo Cedro, CA (US)

(21) Appl. No.: 18/124,726

(22) Filed: Mar. 22, 2023

Related U.S. Application Data
(60) Provisional application No. 63/387,960, filed on Feb. 21, 2023.

(51) Int. Cl. A61B 17/135

(52) U.S. Cl. CPC A61B 17/1355; A61B 2017/00734

(57) ABSTRACT

A battery powered cordless pneumatic tourniquet is disclosed, including a universal cuff extending from an adjustable clamp. The universal cuff comprises a plurality of teeth extending along a length of the universal cuff, the plurality of teeth to permit the adjustable locking with a plurality of complimentary teeth provided on the adjustable clamp. A battery provides power to a pump to selectively, via a microprocessor, expand and contract one or more inflatable components positioned within a housing and apply sufficient pressure to occlude a portion of a patient's anatomy. A central control unit is in communication with a sensor to detects that the pulse of an artery is no longer detectable and instructs the pump that inflates the inflatable components to stop increasing pressure and maintain a current pressure.

### CROSS-REFERENCE TO RELATED APPLICATIONS

The present application claims priority to U.S. Provisional Application No. 63/387,960 filed Dec. 18, 2022, titled “BATTERY POWERED CORDLESS UNIVERSAL SIZED PNEUMATIC TOURNIQUET” which is hereby incorporated by reference in its entirety.

### TECHNICAL FIELD

Embodiments of the invention relate to tourniquets and more specifically to universally sized battery powered tourniquets.

### BACKGROUND

A tourniquet is a device that is used to apply pressure to the body (often a limb or extremity) to stop the flow of blood. Tourniquets are often used in an emergency, in surgery, and in post-operative rehabilitation. Simple tourniquets can be made from a piece of rope and a stick (or other stiff element). However, these primitive designs are less favorable to newer designs which are safer for the patient.

Surgical tourniquets are present in the arts and take various forms. These devices are most often self-contained mechanical devices available in various sizes which is selected based on the occlusion site, blood pressure, as well as the patient's anatomy. Pneumatic tourniquets are available in the current arts. However, these devices include a cord that attaches to an external compressor and are not universally sized. In such, hospitals must maintain a stock of various sizes, while the tubing is a potential source of infectious agents.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

The embodiments provided herein relate to a battery powered cordless pneumatic tourniquet, including a universal cuff extending from an adjustable clamp. The universal cuff comprises a plurality of teeth extending along a length of the universal cuff, the plurality of teeth to permit the adjustable locking with a plurality of complimentary teeth provided on the adjustable clamp. A battery provides power to a pump to selectively, via a microprocessor, expand and contract one or more inflatable components positioned within a housing and apply sufficient pressure to occlude a portion of a patient's anatomy. A central control unit is in communication with a sensor to detects that the pulse of an artery is no longer detectable and instructs the pump that inflates the inflatable components to stop increasing pressure and maintain a current pressure.

In the current arts, pneumatic tourniquets exist which have a cord connecting to an external compressor. While effective, these devices cause numerous drawbacks. For example, the cord must be considered throughout an operation as it may disturb the operating area, cause infection, become tangled, or may fail due damage to the O-rings or other general mechanical causes. Further, the pneumatic tourniquets on the market are not universally sized, requiring medical facilities or emergency users to carry multiple sizes suitable for a variety of patients and applications. When the needed sized tourniquet is not available a larger or smaller sized tourniquet is used that can increase the risk of complication or failure. Even further, the external compressor causes clutter in the operating room, as well as movement of air when moved, can increase the risk of a surgical site infection (SSI).

Further, in the current arts, tourniquets are inflated to a pressure that is not synchronized with the limb occlusion pressure. This may lead to overinflation of the tourniquet causing nerve damage, myonecrosis, and pain. The embodiments provided herein feature the ability to recognize the limb's occlusion pressure to prevent complication associated with tourniquet pain.

Other objects and advantages of the various embodiments of the present invention will become obvious and intuitive to the reader and it is intended that these objects and advantages are within the scope of the present invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a top plan view of the tourniquet including a universal cuff and adjustable clamp, according to some embodiments;

FIG. 2 illustrates a side elevation view of the tourniquet including a universal cuff and adjustable clamp, according to some embodiments;

FIG. 3 illustrates a front elevation view of the adjustable clamp, according to some embodiments;

FIG. 4 illustrates a perspective view of the tourniquet including a universal cuff and adjustable clamp, according to some embodiments;

FIG. 5 illustrates a perspective view of the tourniquet including a universal cuff and adjustable clamp, according to some embodiments;

FIG. 6 illustrates a rear elevation view of the tourniquet including a universal cuff and adjustable clamp, according to some embodiments;

FIG. 7 illustrates a perspective view of the tourniquet including a universal cuff and adjustable clamp in a narrow configuration, according to some embodiments;

FIG. 8 illustrates a perspective view of the tourniquet including a universal cuff and adjustable clamp in a narrow or wide configuration, according to some embodiments;

FIG. 9 illustrates a rear elevation view of the tourniquet including a universal cuff and adjustable clamp in a narrow or wide configuration, according to some embodiments;

FIG. 10 illustrates an exploded view of the tourniquet including a universal cuff and adjustable clamp in a narrow or wide configuration, according to some embodiments;

FIG. 11 illustrates an exploded view of the tourniquet including a universal cuff and adjustable clamp in a narrow or wide configuration, according to some embodiments;

FIG. 12 illustrates a perspective view of the tourniquet and cut-off feature, according to some embodiments;

FIG. 13 illustrates a perspective view of the tourniquet, according to some embodiments;

FIG. 14 illustrates a top plan view of the tourniquet cuff, according to some embodiments;

FIG. 15 illustrates a perspective view of the tourniquet cuff, according to some embodiments;

FIG. 16 illustrates a side elevation view of the tourniquet cuff, according to some embodiments;

FIG. 17 illustrates a detail view of the tourniquet cuff teeth, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided here relate to a battery powered cordless universally sized tourniquet (hereinafter “tourniquet” or “the tourniquet”) which is applied to a patient to prevent blood loss in surgical or emergency settings. The tourniquet minimizes the risk of infection by removing the need for air tubing to cross or encroach on the sterile operating site. The tourniquet is universally sized to remove the need for the medical facility to stock multiple sizes suitable for each patient and application.

During use, the tourniquet is applied as a universally sized tourniquet using the cuff which operates similar to a zip tie. The cuff is inserted through the adjustable clamp until the proper constrictive pressure is applied and the excess portion of the cuff may be cut-off with a cutting tool (e.g., scissors). The locking mechanism will prevent air from leaking and may also include an emergency release valve in case the cuff needs to be deflated. The housing where the cuff is locked into place will also compose of a very small battery powered air compressor to pneumatically inflate the tourniquet. The pump will be attached to a sensor which will be able to regulate and set the desire pressure of the pump in mmHg. The pressure sensor and circuit board may also include a Bluetooth technology to pair with a separate wall mount. The wall mount with have a touch screen (or other interface such as mechanical buttons, etc.) that enables the tourniquet cuff pressure to be set between a pressure range and also for a time range. The tourniquet cuff will automatically deflate when a tourniquet cuff is a certain distance from the wall mount to prevent inadvertent prolonged inflation.

In some embodiments, the tourniquet may include a mechanical emergency deflate release valve. The mechanical emergency deflate release valve is provided as a safety feature which can be used in the event the communication between the computing system and cuff is compromised and the tourniquet should be deflated sooner than when a proximity safety feature is triggered.

In some embodiments, the tourniquet may be used for various occlusion procedures including on the arms, hands, legs, and feet of the patient. The tourniquet may also be used for emergency situations with emergency medical technicians and military to prevent blood loss secondary to traumatic injuries of both upper and lower extremity trauma.

The various medical specialties which may benefit from this device include but are not limited to general surgery, orthopedics, plastic surgery, vascular surgery, and the like. One skilled in the arts will readily understand that additional medical specialties may find the embodiments described herein useful during various procedures.

In some embodiments, the battery powered feature of the tourniquet allows for the avoidance of using any cords that enter or come close to the surgical area or environment. This is because SSI's are a major cost to hospitals and the government.

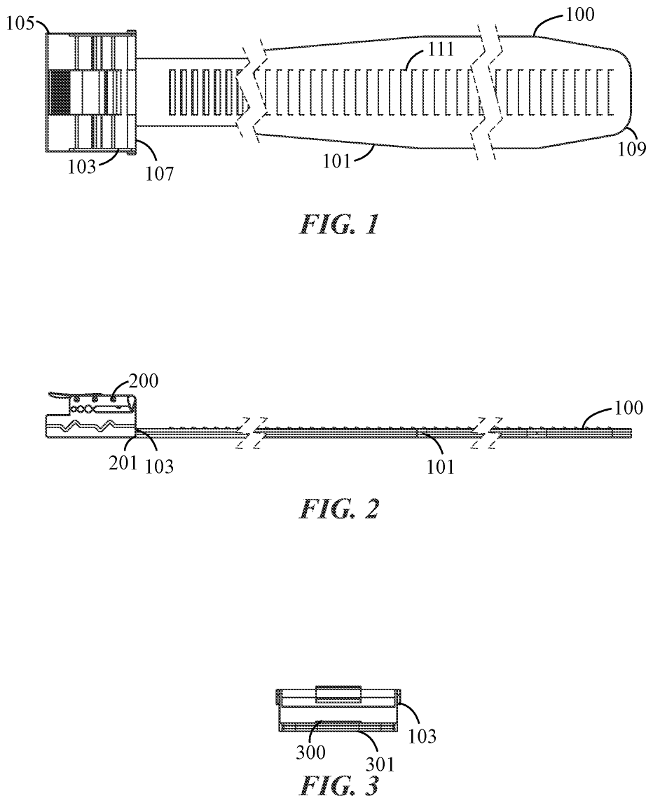

FIG. 1 illustrates a top plan view of the tourniquet 100 including a universal cuff 101 and adjustable clamp 103. The adjustable clamp 103 is positioned at a first end 105 of the tourniquet 100 while the universal cuff 101 extends from a right side 107 of the adjustable clamp 103 to a second end 109 of the tourniquet 100. A plurality of teeth 111 are positioned on a central portion 113 of the universal cuff 101 to allow for the selective adjustability of the tourniquet 100 during use. The plurality of teeth 111 are engaged with a receiver within the adjustable clamp 103 to hold the tourniquet 100 in position during use while maintaining sufficient pressure on the occlusion site on the patient's body.

As illustrated, the narrow and wide configuration of the tourniquet depicts the capability of the tourniquet to not only be universal in length, but in width as well. The tourniquet will have the capability to be one size fits all in length but will also be able to adjust in width in order to respond to the length of the tourniquet that is actually used and inflated. The purpose the narrow and wide configuration is to achieve a maximum possible tourniquet width without interfering with the surgical field. The wider the tourniquet width the lower the inflation pressure needs to be. The lower the inflation pressure the longer the tourniquet can be inflated which lowers the risk of iatrogenic injury.

FIG. 2 illustrates a side elevation view of the tourniquet 100 including a universal cuff 101 and adjustable clamp 103. The adjustable clamp 103 includes a housing 200 wherein the cuff is locked into place will also compose of a very small battery powered air compressor to pneumatically inflate the tourniquet. The pump will be attached to a sensor which will be able to regulate and set the desire pressure of the pump in mmHg. The universal cuff 101 is engaged with a bottom portion 201 of the adjustable clamp 103.

FIG. 3 illustrates a front elevation view of the adjustable clamp 103. The adjustable clamp includes a locking portion 300 and a receiving portion 301. The receiving portion 301 receives the of the universal cuff which is inserted therein until a sufficient pressure is applied to the body of the patient. Once inserted, the locking portion 300 locks the universal cuff in position by engaging with the teeth of the universal cuff prior to the inflation of the tourniquet.

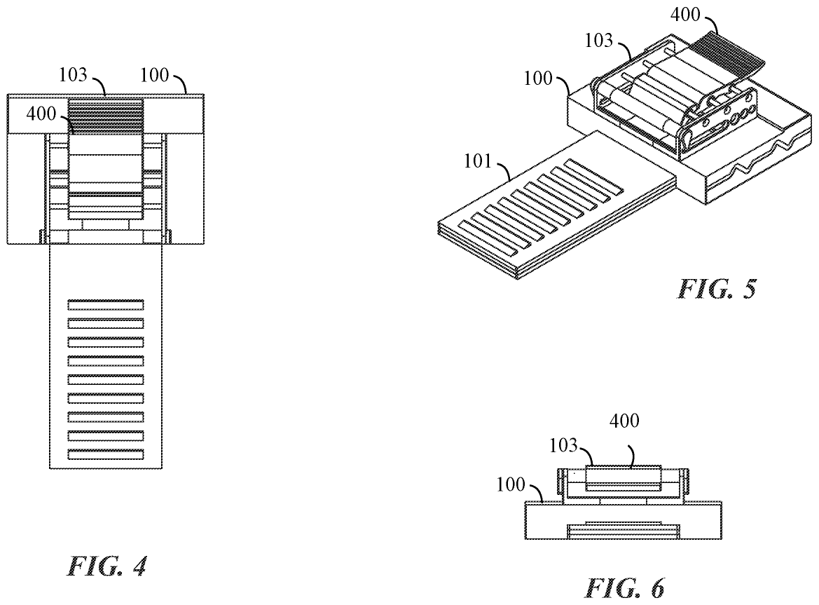

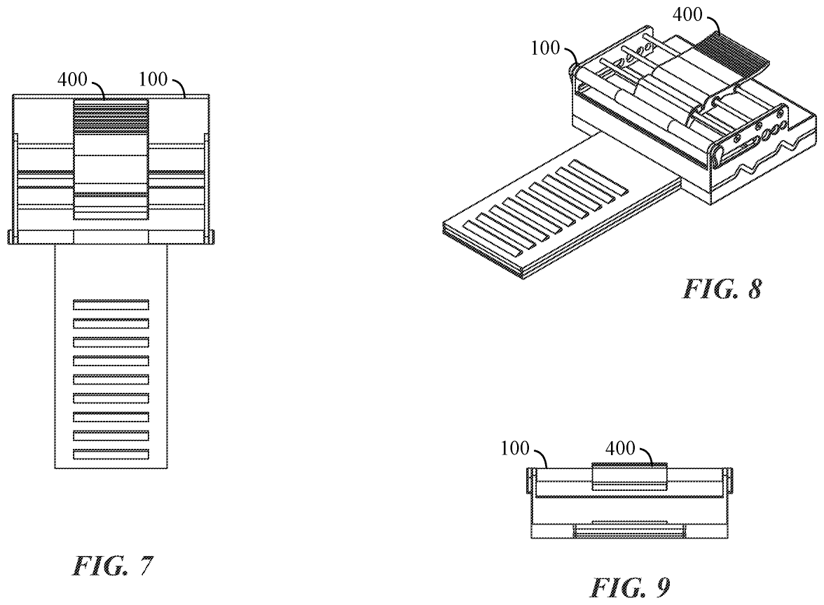

FIGS. 4-6 illustrate a perspective view of the tourniquet 100 including a universal cuff 101 and adjustable clamp 103. The adjustable clamp 103 includes a latch 400 to permit the user to selectively secure and/or release the universal cuff 101 from the adjustable clamp 103 during use. A cuff lock locks the latch 400 in position such that pressure is maintained without the adjustable clamp 103 and universal cuff 101 becoming disengaged. The tourniquet 100 may include a wide and narrow configuration which is illustrated in FIGS. 7-9.

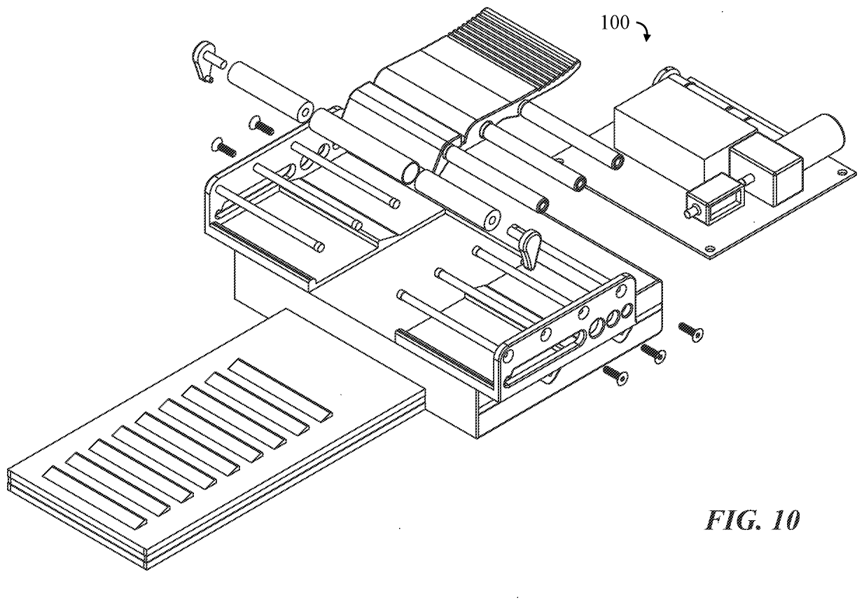

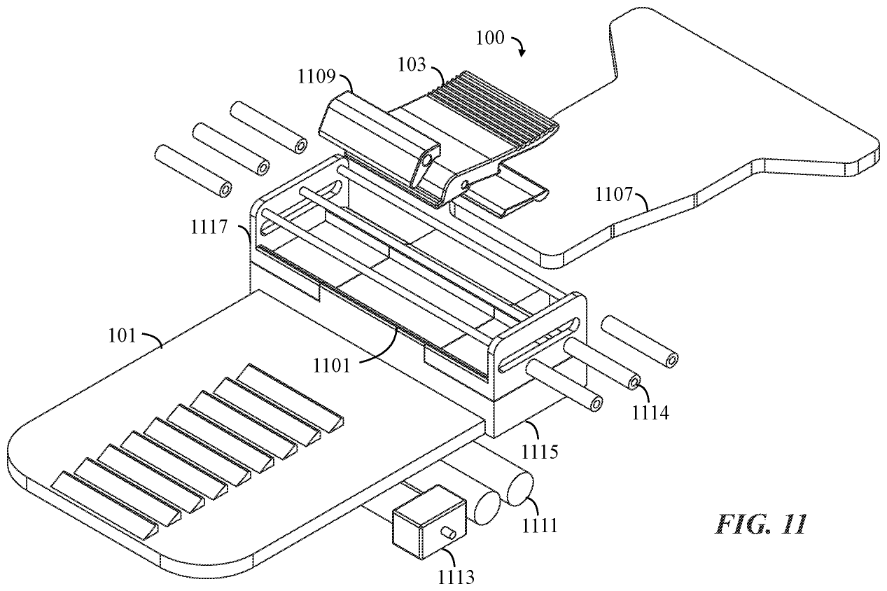

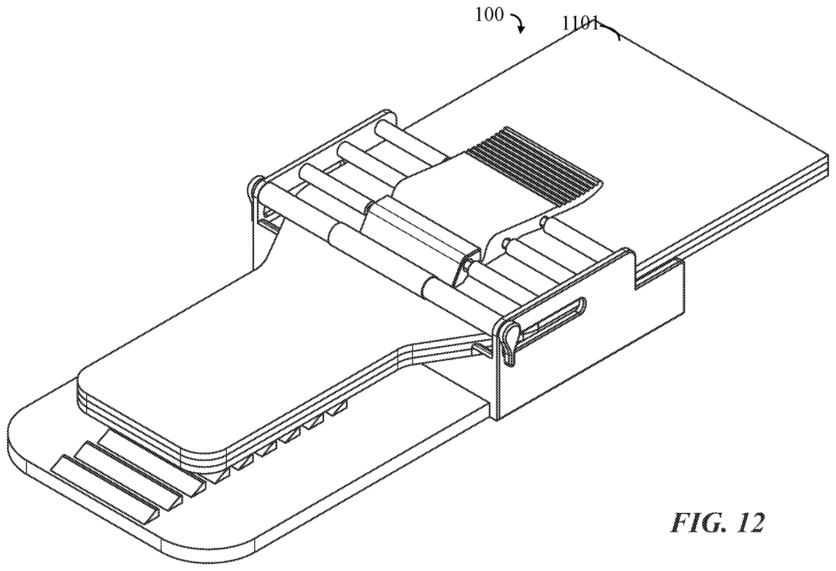

FIG. 10 illustrates an exploded view of the tourniquet 100 including a universal cuff and adjustable clamp in a narrow or wide configuration. FIG. 11 illustrates an exploded view of the tourniquet 100 including a universal cuff 101 and adjustable clamp 103. A universal cuff receiver 1101 includes various widths to accommodate the narrow or wide configurations. The cuff latch 400 in attached to a cuff latch release 1107 to permit the user to selectively engage and release the universal cuff 101 from the adjustable clamp 103 via the cuff lock 1109. A pump 1111 is in electrical communication with one or more batteries 1113 (or similar power supply) to provide power to the pump 1111 during use. The pump 1111 provides air to one or more expandable components 1114 positioned on a first side 1115 and a second side 1117 of the housing 200 of the adjustable clamp 103. The pump 1111 is in communication with at least one sensor to sense the pressure (preferable in mmHg) once the tourniquet is applied to the patient and the pump inflates the inflatable components 1114.

In some embodiments, the tourniquet cuff will have a sensor built into it that can detect the pulse of the artery distal to the bladder of the tourniquet. The sensor may be connected to a microprocessor/computing system. When the sensor detects that the pulse of the artery is no longer detectable the pump that inflates the bladder will stop and maintain that pressure. The sensor may continuously operate and interface with the tourniquet pump until the tourniquet is turned off or manually deflated. The sensor that is likely to be used is a photoplethysmography sensor, however other sensors may be utilized if found to be more suitable. Further, the sensor may detect the limb occlusion pressure, defined as the tourniquet inflation pressure necessary to occlude blood perfusion to the limb. The ability for a tourniquet to function specific to the limb occlusion pressure will allow for optimal inflation pressure preventing over inflation which can cause iatrogenic injury. The tourniquets built in sensor will not require a separate device to be used distal to the tourniquet on a finger or toe to detect when the occlusion pressure has been achieved. The sensor can be overriding allowing the user to manually enter the desired tourniquet pressure and time. This will be achieved via the external central control unit (wall mount). The external central control unit will be wirelessly connected to the internal tourniquet cuff microprocessor/computing system via a Bluetooth technology or variation thereof.

FIG. 12 illustrates a perspective view of the tourniquet 100 and cut-off feature. The tourniquet cut off feature is provided to remove the excess portion of the universally sized cuff. The excess material will be cut off with a medical utility scissor, or similar cutting tool. The purpose of removing the excess material is to avoid violating the sterile field and interference with the surgical site. The cut off feature will not interfere with maintaining the bladder pressure because once the tourniquet is fastened via the locking mechanism, the bladder will be sealed preventing air leakage from the excess cut off portion.

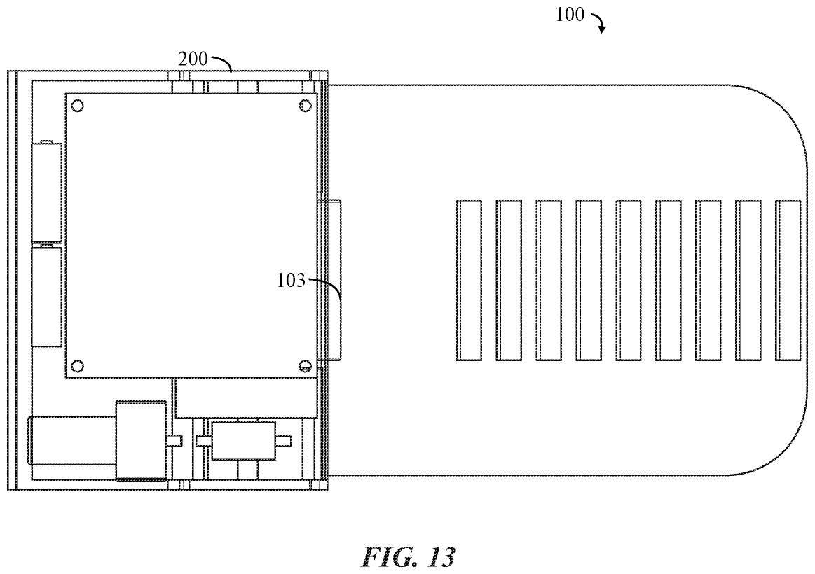

FIG. 13 illustrates a perspective view of the tourniquet 100 and internal components provided within the housing 200 of the adjustable clamp 103.

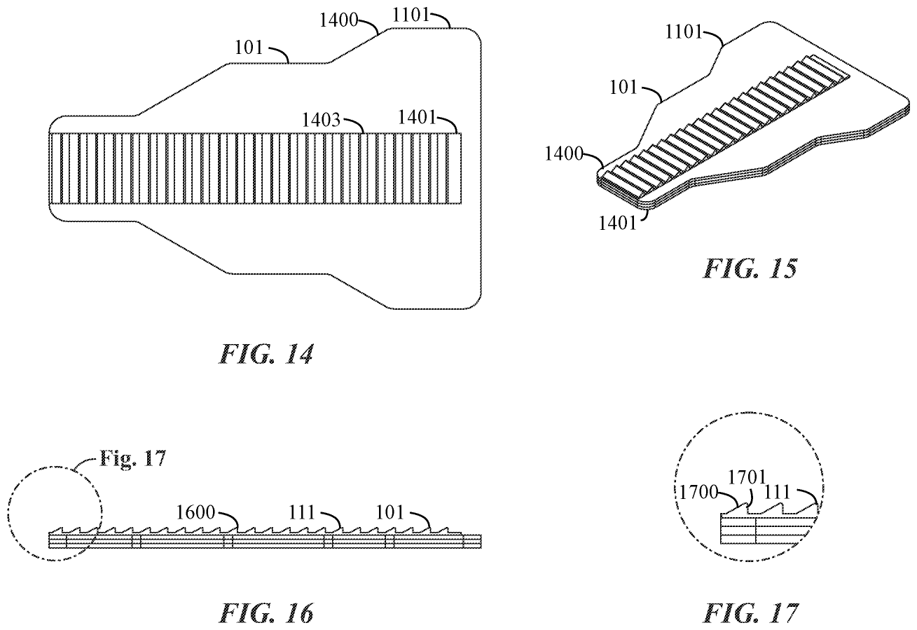

FIG. 14 and FIG. 15 illustrate the tourniquet's universal cuff receiver 1101 comprising a flexible material between each of a first layer 1400 and third layer 1401. A vinyl over-layer (not shown) is also provided. A second complimentary set of teeth 1403 engage with the plurality of teeth on the universal cuff 101.

FIG. 16 illustrates a side elevation view of the tourniquet's universal cuff 101. The plurality of teeth 101 are positioned on the top side 1600 of the universal cuff 101 to engage with the locking mechanism of the adjustable clamp as well as the teeth of the universal cuff. FIG. 17 illustrates a detail view of the tourniquet cuff and the plurality of teeth 111. Each of the plurality of teeth 111 include a beveled portion 1700 and a vertical portion 1701 to permit the uni-directional movement of the universal cuff 101 within the adjustable clamp.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A battery powered cordless pneumatic tourniquet, comprising:
a universal cuff extending from an adjustable clamp, the universal cuff comprising a plurality of teeth extending along a length of the universal cuff, the plurality of teeth to permit the adjustable locking with a plurality of complimentary teeth provided on the adjustable clamp; and
a battery to provide power to a pump, the pump to selectively, via a microprocessor, expand and contract one or more inflatable components positioned within a housing and apply sufficient pressure to occlude a portion of a patient's anatomy.

2. The battery powered cordless pneumatic tourniquet of claim 1, wherein the universal cuff includes a top surface whereon the plurality of teeth are provided.

3. The battery powered cordless pneumatic tourniquet of claim 1, the universal cuff extends into a right side of the adjustable clamp.

4. The battery powered cordless pneumatic tourniquet of claim 1, wherein the universal cuff is engaged with a bottom portion of the adjustable clamp.

5. The battery powered cordless pneumatic tourniquet of claim 1, universal clamp includes a locking mechanism.

6. The battery powered cordless pneumatic tourniquet of claim 5, wherein the adjustable clamp includes a latch to permit the selective opening and closing of the locking mechanism.

7. The battery powered cordless pneumatic tourniquet of claim 1, further comprising a cut off feature to remove the excess portion of the universal cuff.

8. The battery powered cordless pneumatic tourniquet of claim 1, wherein a housing contains the battery, the pump, and the one or more inflatable components.

9. The battery powered cordless pneumatic tourniquet of claim 1, further comprising a cut off feature to remove the excess portion of the universally sized cuff.

10. A battery powered cordless pneumatic tourniquet, comprising:
a universal cuff extending from an adjustable clamp, the universal cuff comprising a plurality of teeth extending along a length of the universal cuff, the plurality of teeth to permit the adjustable locking with a plurality of complimentary teeth provided on the adjustable clamp;
a battery to provide power to a pump, the pump to selectively, via a microprocessor, expand and contract one or more inflatable components positioned within a housing and apply sufficient pressure to occlude a portion of a patient's anatomy; and
a central control unit in communication with a sensor, wherein the sensor detects that the pulse of an artery is no longer detectable and instructs the pump that inflates the inflatable components to stop increasing pressure and maintain a current pressure.

11. The battery powered cordless pneumatic tourniquet of claim 10, wherein the universal cuff includes a top surface whereon the plurality of teeth are provided.

12. The battery powered cordless pneumatic tourniquet of claim 11, the universal cuff extends into a right side of the adjustable clamp.

13. The battery powered cordless pneumatic tourniquet of claim 12, wherein the universal cuff is engaged with a bottom portion of the adjustable clamp.

14. The battery powered cordless pneumatic tourniquet of claim 13, universal clamp includes a locking mechanism.

15. The battery powered cordless pneumatic tourniquet of claim 14, wherein the adjustable clamp includes a latch to permit the selective opening and closing of the locking mechanism.

16. The battery powered cordless pneumatic tourniquet of claim 5, further comprising a cut off feature to remove the excess portion of the universal cuff.

17. The battery powered cordless pneumatic tourniquet of claim 16, wherein a housing contains the battery, the pump, and the one or more inflatable components.

18. The battery powered cordless pneumatic tourniquet of claim 17, further comprising a cut off feature to remove the excess portion of the universally sized cuff.
